(19) United States
(12) Patent Application Publication
(10) Pub. No.: US 2012/0245180 A1
(43) Pub. Date: Sep. 27, 2012

(54) COMBINATION

(75) Inventors: Kurt R. Auger, Collegeville, PA (US); Kurtis Earl Bachman, Collegeville, PA (US); Tona M. Gilmer, Research Triangle Park, NC (US); James G. Greger, JR., Collegeville, PA (US); Joel David Greshock, Collegeville, PA (US); Sylvie Laquerre, King of Prussia, PA (US); Li Liu, King of Prussia, PA (US); Shannon Renae Morris, Research Triangle Park, NC (US)

(73) Assignee: GlaxoSmithKline LLC, Philadelphia, PA (US)

(21) Appl. No.: 13/498,381

(22) PCT Filed: Sep. 28, 2010
(86) PCT No.: PCT/US10/50495

§ 371 Date: Jun. 11, 2012

Related U.S. Application Data
(60) Provisional application No. 61/246,265, filed on Sep. 28, 2009.
(60) Provisional application No. 61/385,738, filed on Sep. 23, 2010.

(51) Int. Cl. A61K 31/519; A61P 35/00

(52) U.S. Cl. 514/252.02

(57) ABSTRACT

The present invention relates to a method of treating cancer in a mammal and to pharmaceutical combinations useful in such treatment. In particular, the method relates to a novel combination comprising the MEK inhibitor: N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and the PI3 kinase inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, pharmaceutical compositions comprising the same, and methods of using such combinations in the treatment of cancer.

### FIELD OF THE INVENTION

The present invention relates to a method of treating cancer in a mammal and to combinations useful in such treatment. In particular, the method relates to a novel combination comprising the MEK inhibitor: N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and the PI3K inhibitor: 2,4-difluoro-N-{2-(methyloxy)-5-[4-(4-pyridazinyl)-6-quinolinyl]-3-pyridinyl}benzenesulfonamide, or a pharmaceutically acceptable salt thereof, pharmaceutical compositions comprising the same, and methods of using such combinations in the treatment of cancer.

### BACKGROUND OF THE INVENTION

Effective treatment of hyperproliferative disorders including cancer is a continuing goal in the oncology field. Generally, cancer results from the deregulation of the normal processes that control cell division, differentiation and apoptotic cell death. Apoptosis (programmed cell death) plays essential roles in embryonic development and pathogenesis of various diseases, such as degenerative neuronal diseases, cardiovascular diseases and cancer. One of the most commonly studied pathways, which involves kinase regulation of apoptosis, is cellular signaling from growth factor receptors at the cell surface to the nucleus (Crews and Erikson, Cell, 74:215-17, 1993).

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity including benign and malignant proliferative disorders as well as diseases resulting from inappropriate activation of the immune and nervous systems. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases and Protein Serine/Threonine Kinases, based on the amino acid residue they phosphorylate. The protein serine/threonine kinases (PSTK), includes cyclic AMP- and cyclic GMP-dependent protein kinases, calcium and phospholipid dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also in progress to identify modulators of tyrosine kinases as well.

Mitogen-activated protein kinase (MAPK) Kinase/extracellular signal-regulated kinase (ERK) kinase (hereinafter referred to as MEK) is known to be involved in the regulation of numerous cellular processes. The Raf family (B-Raf, C-Raf etc.) activates the MEK family (MEK-1, MEK-2 etc.) and the MEK family activates the ERK family (ERK-1 and ERK-2). Broadly, the signaling activity of the RAF/MEK/ERK pathway controls mRNA translation. This includes genes related to the cell cycle. Hence, hyperactivation of this pathway can lead to uncontrolled cell proliferation. Deregulation of the RAF/MEK/ERK pathway by ERK hyperactivation is seen in approximately 30% of all human malignancies (Allen, L F, et al. Semin. Oncol. 2003. 30(5 Suppl 16):105-16). RAS, which can signal through both the PI3K/AKT and RAF/MEK/ERK, has a mutated oncogenic protein in 15% of all cancers (Davies, H. et al. Nature. 2002. 417:949-54). Also, activating BRAF mutations have been identified at a high frequency in specific tumor types (e.g., melanomas) (Davies, H. et al. Nature. 2002. 417:949-54). Although activating mutations in MEK itself don't appear to frequently occur in human cancers, MEK is thought to be an important drug target for treating human cancer because of its central role in the ERK pathway. Further, MEK inhibitory activity effectively induces inhibition of ERK1/2 activity and suppression of cell proliferation (The Journal of Biological Chemistry, vol. 276, No. 4, pp. 2686-2692, 2001), and the compound is expected to show effects on diseases caused by undesirable cell proliferation, such as tumor genesis and/or cancer.

The phosphoinositide 3-kinase (PI3K) pathway is among the most commonly activated pathways in human cancer. The function and importance of this pathway in tumorigenesis and tumor progression is well established (Samuels & Ericson. Curr. Opp in Oncology, 2006. 18: 77-82). PI3K-AKT signaling appears to be a pivotal modulator of cell survival, proliferation and metabolism. This includes the activation of mammalian target of rapamycin (mTOR), a PI3K protein family member and direct regulator of cell growth and translation. Thus, the deregulation of PI3K/AKT/mTOR signaling in tumors contributes to a cellular phenotype that demonstrates numerous hallmarks of malignancies, which includes unlimited reproductive potential and the evasion of apoptosis (Hanahan & Weinberg, Cell. 2000. 100:57-70).

The PI3K family consists of 15 proteins that share sequence homology, particularly within their kinase domains; however; they have distinct substrate specificities and modes of regulation (Vivanco & Sawyers. Nat. Rev. Cancer, 2002.2:489-501). Class I PI3-kinases phosphorylate inositol-containing lipids, known as phosphatidylinositols (PtdIns) at the 3 position. The primary substrate of Class I family members, PtdIns-4,5-P2 (PIP2) is converted to PtdIns-3,4,5-P3 (PIP3) by these kinases. PIP3 is a critical second messenger which recruits proteins that contain pleckstrin homology domains to the cell membrane where they are activated. The most studied of these proteins is AKT which promotes cell survival, growth, and proliferation. Upon activation, AKT moves to the cytoplasm and nucleus where it phosphorylates numerous substrates, including mTOR (TORC1). In addition to AKT, PI3K activates other pathways that are implicated in carcinogenesis such as PDK1, CDC42 and RAC1 (Samuels & Ericson. Curr. Opp in Oncology, 2006. 18: 77-82).

In the study of human tumors, activation of the PI3K/AKT/mTOR signaling pathway can occur via numerous mechanisms. Genetic deregulation of the pathway is common and can occur in a number of ways (reviewed in Samuels & Ericson. Curr. Opp in Oncology, 2006. 18: 77-82). Activating mutations of the PIK3CA gene (coding for the p110α catalytic subunit of PI3K) occur in a significant percentage of human tumors including breast, ovarian, endometrial, and colorectal cancer. Activating DNA amplifications of this gene also occur less frequently in a number of different tumor types. Mutations in the p85a regulatory subunit of PI3K (PIK3R1), which are thought to disrupt the C2-iSH2 interaction between PIK3R1 and PIK3CA, occur in ovarian, glioblastoma and colorectal cancer. The tumor suppressor PTEN, which dephosphorylates PIP3 to generate PIP2 and thus acts as an inhibitor of the PI3K pathway, is commonly mutated, deleted, or epigenetically silenced. Finally, the pathway can also be genetically activated downstream of PI3K by DNA amplification or mutation of AKT; however these genetic events occur much less frequently in human cancer. Inhibiting PI3K isoforms, particularly PI3Kα, are known to be useful in the treatment of cancer (see for example WO 05/121142, WO 08/144,463, WO 08/144,464, WO 07/136,940).

### SUMMARY OF THE INVENTION

One embodiment of this invention provides a combination comprising:

(i) a compound of Structure (I):

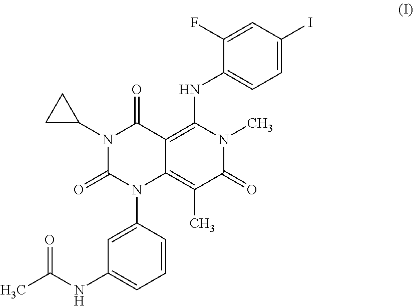

N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide (hereinafter Compound A)
or a pharmaceutically acceptable salt thereof; and
(ii) a compound of Structure (II):

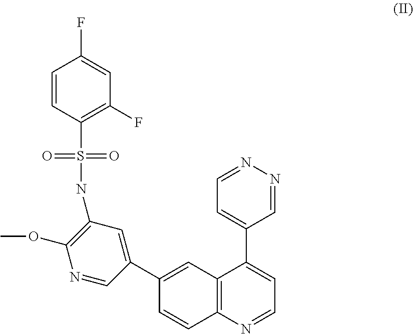

### 2,4-difluoro-N-{2-(methyloxy)-5-[4-(4-pyridazinyl)-6-quinolinyl]-3-pyridinyl}benzenesulfonamide (hereinafter Compound B)

or a pharmaceutically acceptable salt thereof.

One embodiment of this invention provides a method of treating cancer in a human in need thereof which comprises the in vivo administration of a therapeutically effective amount of a combination of Compound A, or a pharmaceutically acceptable salt or solvate, suitably the dimethyl sulfoxide solvate, thereof, and Compound B, or a pharmaceutically acceptable salt thereof, to such human.

One embodiment of this invention provides a method of treating cancer in a human in need thereof which comprises the in vivo administration of a therapeutically effective amount of a combination of Compound A, or a pharmaceutically acceptable salt or solvate, suitably the dimethyl sulfoxide solvate, thereof, and Compound B, or a pharmaceutically acceptable salt thereof, to such human,
wherein the combination is administered within a specified period, and
wherein the combination is administered for a duration of time.

One embodiment of this invention provides a method of treating cancer in a human in need thereof which comprises the in vivo administration of a therapeutically effective amount of a combination of Compound A, or a pharmaceutically acceptable salt or solvate, suitably the dimethyl sulfoxide solvate, thereof, and Compound B, or a pharmaceutically acceptable salt thereof, to such human,
Wherein compounds A and B are administered sequentially.

### DETAILED DESCRIPTION OF THE INVENTION

The present invention relates to combinations that exhibit antiproliferative activity. Suitably, the method relates to methods of treating cancer by the co-administration of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide (Compound A), or a pharmaceutically acceptable salt or solvate, suitably the dimethyl sulfoxide solvate thereof, which compound is represented by Structure I:

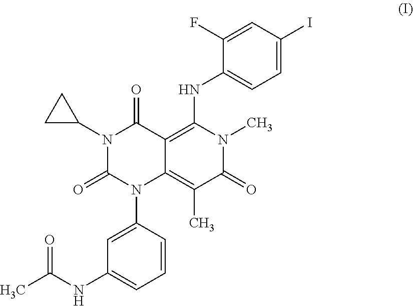

and 2,4-difluoro-N-{2-(methyloxy)-5-[4-(4-pyridazinyl)-6-quinolinyl]-3-pyridinyl}benzenesulfonamide (Compound B), or a pharmaceutically acceptable salt thereof; which compound is represented by the following structure

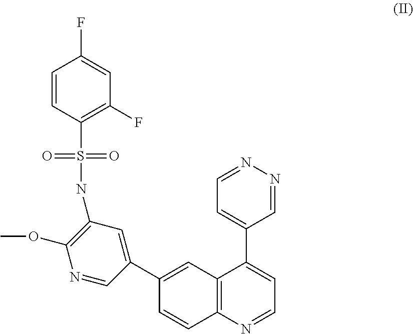

Compound A, also known as N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide is disclosed and claimed, along with pharmaceutically acceptable salts and solvates thereof, as being useful as an inhibitor of MEK activity, particularly in treatment of cancer, in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference, Compound A is the compound of Example 4-1. Compound A can be prepared as described in International Application No. PCT/JP2005/011082. Compound A can be prepared as described in United States Patent Publication No. US 2006/0014768, Published Jan. 19, 2006, the entire disclosure of which is hereby incorporated by reference.

Suitably, Compound A is in the form of a dimethyl sulfoxide solvate. Suitably, Compound A is in the form of a sodium salt. Suitably, Compound A is in the form of a solvate selected from: hydrate, acetic acid, ethanol, nitromethane, chlorobenzene, 1-pentanol, isopropyl alcohol, ethylene glycol and 3-methyl-1-butanol. These solvates and salt forms can be prepared by one of skill in the art from the description in International Application No. PCT/JP2005/011082 or United States Patent Publication No. US 2006/0014768.

Compound B is disclosed and claimed, along with pharmaceutically acceptable salts thereof, as being useful as an inhibitor of PI3K activity, particularly in treatment of cancer, in International Application No. PCT/US2008/063819, having an International filing date of May 16, 2008; International Publication Number WO 2008/144463 and an International Publication date of Nov. 27, 2008, the entire disclosure of which is hereby incorporated by reference, Compound B is the compound of example 345. Compound B can be prepared as described in International Application No. PCT/US2008/063819.

Suitably, Compound B is in the form of free base.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Examples of suitable pharmaceutically acceptable solvents include, without limitation, water, dimethyl sulfoxide, ethanol and acetic acid. Suitably the solvent used is water.

The pharmaceutically acceptable salts of the compounds of the invention are readily prepared by those of skill in the art.

By the term “treating” and derivatives thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

By the term “periodically administration” or variations thereof, is meant that the drug is not administered to the human with drug holidays. A drug holiday (sometimes also called a drug vacation, medication vacation, structured treatment interruption or strategic treatment interruption) is when a patient stops taking a medication(s) for a period of time; anywhere from a few days to several months

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and derivatives thereof, as used herein is meant either simultaneous administration or any manner of separate sequential administration of a therapeutically effective amount of Compound A, or a pharmaceutically acceptable salt or solvate thereof, and Compound B or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable salt or solvate thereof, and Compound B, or a pharmaceutically acceptable salt thereof, according to the invention. When both compounds are administered simultaneously, the combination kit can contain Compound A, or a pharmaceutically acceptable salt or solvate thereof, and Compound B, or a pharmaceutically acceptable salt thereof, in a single pharmaceutical composition, such as a tablet, or in separate pharmaceutical compositions. When the compounds are not administered simultaneously, the combination kit will contain Compound A, or a pharmaceutically acceptable salt or solvate thereof, and Compound B, or a pharmaceutically acceptable salt thereof, in separate pharmaceutical compositions. The combination kit can comprise Compound A, or a pharmaceutically acceptable salt or solvate thereof, and Compound B, or a pharmaceutically acceptable salt thereof, in separate pharmaceutical compositions in a single package or in separate pharmaceutical compositions in separate packages.

In one aspect there is provided a combination kit comprising the components:
Compound A, or a pharmaceutically acceptable salt or solvate thereof, in association with a pharmaceutically acceptable carrier; and
Compound B, or a pharmaceutically acceptable salt thereof, in association with a pharmaceutically acceptable carrier.
In one embodiment of the invention the combination kit comprises the following components:
Compound A, or a pharmaceutically acceptable salt or solvate thereof, in association with a pharmaceutically acceptable carrier; and
Compound B, or a pharmaceutically acceptable salt thereof, in association with a pharmaceutically acceptable carrier,
wherein the components are provided in a form which is suitable for sequential, separate and/or simultaneous administration.
In one embodiment the combination kit comprises:
a first container comprising Compound A, or a pharmaceutically acceptable salt or solvate thereof, in association with a pharmaceutically acceptable carrier; and
a second container comprising Compound B, or a pharmaceutically acceptable salt thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

By the term “triple negative” breast cancer, as used herein is meant any breast cancer that does not express the genes for estrogen receptor (ER), progesterone receptor (PR) or Her2/neu. This subtype of breast cancer is clinically characterised as more aggressive and less responsive to standard treatment and associated poorer overall patient prognosis. It is diagnosed more frequently in younger women, women with BRCA1 mutations, and those belonging to African-American and Hispanic ethnic groups, and those having a recent birth.

A basal-like breast tumor is a subtype of aggressive breast cancer that has a short relapse time. African-American women that are premenopausal are at higher than average risk to develop basal-like breast tumors, which are usually triple-negative for estrogen, progesterone, and HER2 receptors. Basal-like breast tumors may be high grade and diagnosed at a late stage, requiring powerful chemotherapy regimens.

As used herein the term “Compound A²” means—Compound A, or a pharmaceutically acceptable salt or solvate thereof—.

As used herein the term “Compound B²” means—Compound B, or a pharmaceutically acceptable salt thereof—.

Suitably the combinations of this invention are administered within a “specified period”.

By the term “specified period” and derivatives thereof, as used herein is meant the interval of time between the administration of one of Compound A² and Compound B² and the other of Compound A² and Compound B². Unless otherwise defined, the specified period can include simultaneous administration. Unless otherwise defined the specified period refers to administration of Compound A² and Compound B² during a single day.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and derivatives thereof, as used herein is meant that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

### Regarding “Specified Period” Administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and derivates thereof, as used herein is meant that one of Compound A² and Compound B² is administered once a day for two or more consecutive days and the other of Compound A² and Compound B² is subsequently administered once a day for two or more consecutive days. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

### Regarding Sequential Administration:

Suitably, one of Compound A² and Compound B² is administered for from 2 to 30 consecutive days, followed by an optional drug holiday, followed by administration of the other of Compound A² and Compound B² for from 2 to 30 consecutive days. Suitably, one of Compound A² and Compound B² is administered for from 2 to 21 consecutive days, followed by an optional drug holiday, followed by administration of the other of Compound A² and Compound B² for from 2 to 21 consecutive days. Suitably, one of Compound A² and Compound B² is administered for from 2 to 14 consecutive days, followed by a drug holiday of from 1 to 14 days, followed by administration of the other of Compound A² and Compound B² for from 2 to 14 consecutive days. Suitably, one of Compound A² and Compound B² is administered for from 3 to 7 consecutive days, followed by a drug holiday of from 3 to 10 days, followed by administration of the other of Compound A² and Compound B² for from 3 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 0.125 mg to about 10 mg; suitably, the amount will be selected from about 0.25 mg to about 9 mg; suitably, the amount will be selected from about 0.25 mg to about 8 mg; suitably, the amount will be selected from about 0.5 mg to about 8 mg; suitably, the amount will be selected from about 0.5 mg to about 7 mg; suitably, the amount will be selected from about 1 mg to about 7 mg; suitably, the amount will be about 5 mg. Accordingly, the amount of Compound A administered as part of the combination according to the present invention will be an amount selected from about 0.125 mg to about 10 mg. For example, the amount of Compound A² administered as part of the combination according to the present invention can be 0.125 mg, 0.25 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg.

Suitably, the amount of Compound B² administered as part of the combination according to the present invention will be an amount selected from about 0.25 mg to about 75 mg; suitably, the amount will be selected from about 0.5 mg to about 50 mg; suitably, the amount will be selected from about 1 mg to about 25 mg; suitably, the amount will be selected from about 2 mg to about 20 mg; suitably, the amount will be selected from about 4 mg to about 16 mg; suitably, the amount will be selected from about 6 mg to about 12 mg;

suitably, the amount will be about 10 mg. Accordingly, the amount of Compound B² administered as part of the combination according to the present invention will be an amount selected from about 0.5 mg to about 50 mg. For example, the amount of Compound B² administered as part of the combination according to the present invention can be 0.5 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 20 mg, 21 mg, 22 mg, 23 mg, 25 mg, 26 mg, 27 mg, 28 mg, 29 mg, 30 mg, 35 mg, 40 mg, 45 mg, or 50 mg.

As used herein, all amounts specified for Compound A² and Compound B² are indicated as the administered amount of free or unsalted and unsolvated compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The combinations of the present invention are tested for efficacy, advantageous and synergistic properties according to known procedures. Suitably, the combinations of the invention are tested for efficacy, advantageous and synergistic properties generally according to the following combination cell proliferation assays. Cells are plated in 96 or 384-well plates in culture media appropriate for each cell type, supplemented with 10% FBS and 1% penicillin/streptomycin, and incubated overnight at 37° C., 5% CO₂. Cells are treated in a grid manner with dilution of Compound A² (10 dilutions, including no compound, of 3-fold dilutions starting from 0.250-20 μM depending of compound) and also treated with Compound B² (10 dilutions, including no compound, of 3-fold dilutions starting from 0.150-20 μM depending of compound) and incubated as above for a further 72 hours. In some instances compounds are added in a staggered manner and incubation time can be extended up to 7 days. Cell growth is measured using CellTiter-Glo® reagent according to the manufacturer's protocol and signals are read on a PerkinElmer EnVision™ reader set for luminescence mode with a 0.5-second read. Data are analyzed as described below.

Results are expressed as a percentage of the t=0 value and plotted against compound(s) concentration. The t=0 value is normalized to 100% and represents the number of cells present at the time of compound addition. The cellular response is determined for each compound and/or compound combination using a 4- or 6-parameter curve fit of cell viability against concentration using the IDBS XLfit plug-in for Microsoft Excel software and determining the concentration required for 50% inhibition of cell growth (gIC₅₀). Background correction is made by subtraction of values from wells containing no cells. For each drug combination a Combination Index (CI), Excess Over Highest Single Agent (EOHSA) and Excess Over Bliss (EOBliss) are calculated according to known methods such as described in Chou and Talalay (1984) Advances in Enzyme Regulation, 22, 37 to 55; and Berenbaum, MC (1981) Adv. Cancer Research, 35, 269-335.

Because the combinations of the present invention are active in the above assays they exhibit advantageous therapeutic utility in treating cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,

malignant lymphoma, hodgkins lymphoma, non-hodgkins lymphoma, lymphoblastic T cell lymphoma, Burkitt's lymphoma, follicular lymphoma,

neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, liver, colon, breast, pancreatic and prostate.

Suitably, the present invention relates to a method for treating or lessening the severity of a cancer selected from breast, liver, lung, pancreatic, and colon.

Suitably, the present invention relates to a method of treating or lessening the severity of a cancer that is either wild type or mutant for certain biomarker(s).

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

Cancers that are either wild type or mutant for biomarker(s) and either wild type or mutant for PI3K/Pten are identified by known methods.

V-Ki-ras2 Kirsten rat sarcoma viral oncogene homolog, also known as KRAS, is a protein which in humans is encoded by the KRAS gene. Like other members of the Ras family, the KRAS protein is a GTPase and is an early player in many signal transduction pathways. KRAS is usually tethered to cell membranes because of the presence of an isoprenyl group on its C-terminus. When mutated, KRAS is an oncogene. The protein product of the normal KRAS gene performs an essential function in normal tissue signaling, and the mutation of a KRAS gene is an essential step in the development of many cancers.

The N-ras oncogene is a member of the RAS gene family. It is mapped on chromosome 1, and it is activated in HL60, a promyelocytic leukemia line. The order of nearby genes is as follows: cen—CD2—NGFB—NRAS—tel. The mammalian ras gene family consists of the harvey and kirsten ras genes (c-Hras1 and c-Kras2), an inactive pseudogene of each (c-Hras2 and c-Kras1) and the N-ras gene. They differ significantly only in the C-terminal 40 amino acids. These ras genes have GTP/GDP binding and GTPase activity, and their normal function may be as G-like regulatory proteins involved in the normal control of cell growth. Mutations which change amino acid residues 12, 13 or 61 activate the potential of N-ras to transform cultured cells and are implicated in a variety of human tumors. The N-ras gene specifies two main transcripts of 2 Kb and 4.3 Kb. The difference between the two transcripts is a simple extension through the termination site of the 2 Kb transcript. The N-ras gene consists of seven exons (-I, I, II, III, IV, V, VI). The smaller 2 Kb transcript contains the Vla exon, and the larger 4.3 Kb transcript contains the Vlb exon which is just a longer form of the Vla exon. Both transcripts encode identical proteins as they differ only the 3′ untranslated region. The sequence of the shorter 2 Kb transcript is presented here. The 4.3 Kb transcript sequence is not available.

Wild type or mutant Ras/Raf or PI3K/PTEN tumor cells can be identified by DNA amplification and sequencing techniques, DNA and RNA detection techniques, including, but not limited to Northern and Southern blot, respectively, and/or various biochip and array technologies. This can include cytogenetic aberrations and transcript abundance. Wild type and mutant polypeptides can be detected by a variety of techniques including, but not limited to immunodiagnostic techniques such as ELISA, Western blot or immunocyto chemistry.

This invention provides a combination comprising N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides for a combination comprising N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, for use in therapy.

This invention also provides for a combination comprising N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, for use in treating cancer.

This invention also provides a pharmaceutical composition comprising a combination of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides a combination kit comprising N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof.

This invention also provides for the use of a combination comprising N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, in the manufacture of a medicament.

This invention also provides for the use of a combination comprising N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, in the manufacture of a medicament to treat cancer.

This invention also provides a method of treating cancer which comprises administering a combination of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, to a subject in need thereof.

This invention also relates to a method of treating cancer, which comprises administering a combination of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and 2,4-difluoro-N-{2-(methyloxy)-5-[4-(4-pyridazinyl)-6-quinolinyl]-3-pyridinyl}benzenesulfonamide, or a pharmaceutically acceptable salt thereof, to a subject in need thereof, wherein the amount of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof is selected from about 0.5 mg to about 3 mg and the amount of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof is selected from about 0.5 mg to about 3 mg.

The following examples are intended for illustration only and are not intended to limit the scope of the invention in any way.

### Experimental Details

### Preparation of MEK Inhibitors

MEK inhibitors which are suitable for use in the present combinations, particularly N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide dimethyl sulfoxide,

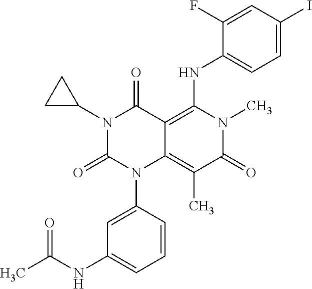

can be prepared according to International Patent Publication No. WO2005/121142.
PI3K inhibitors which are suitable for use in the present combinations, particularly 2,4-difluoro-N-{2-(methyloxy)-5-[4-(4-pyridazinyl)-6-quinolinyl]-3-pyridinyl}benzenesulfonamide

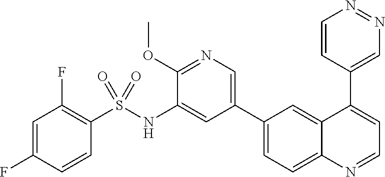

can be prepared according to International Patent Publication No. WO08/144,463 (Example 345)
Compound A as described in the Experimental section refers to the dimethyl sulfoxide solvate of N-{3-[3-cyclopropyl-5-(2-fluoro-4-iodo-phenylamino)6,8-dimethyl-2,4,7-trioxo-3,4,6,7-tetrahydro-2H-pyrido[4,3-d]pyrimidin-1-yl]phenyl}acetamide.
In Vitro Cell Growth Inhibition and Apoptosis Induction by Compound A, Compound B and their Combination in Tumor Cell Lines

### Study #1. Colon, Lung and Pancreatic Cancer Cell Lines

### Experimental Preparation(s)

Combination drug tests with Compounds A and B were conducted using a panel of cell lines from human colon cancers (n=26), lung cancers (n=14) and pancreatic cancers (n=6) (Table 1). Cell lines were purchased commercially [from ATCC (Manassas, Va., USA) or DSMZ (Braunschweig, Germany)] and grown in RPMI-1640 supplemented with 2 mM glutamine, 1 mM sodium pyruvate and 10% fetal bovine serum (except for Capan-1 and HuP-T4 which were grown with 20% fetal bovine serum) and maintained at 37° C. and 5% CO₂ in a humid incubator.

### Experimental Protocol(s)

### Fixed Ratio Drug Combination Assay

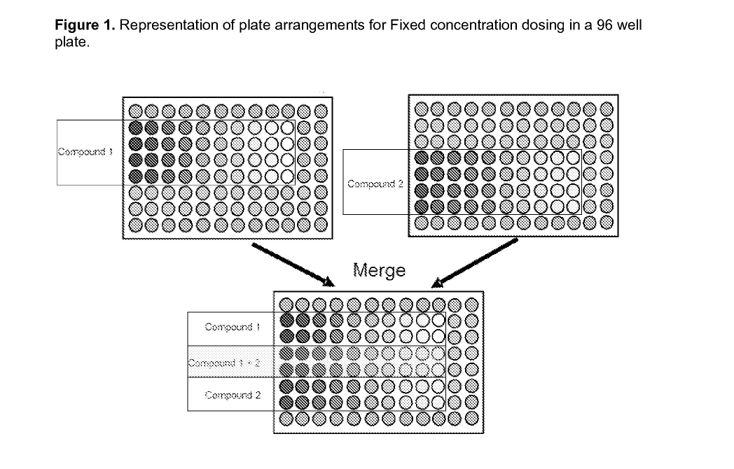

The dilution design of the Fixed Ratio Drug Combination Assay can be seen in FIG. 1. First, the test compounds were prepared as 10 mM stocks in 100% dimethyl sulfoxide (DMSO). Further dilutions of the compounds were made with DMSO. The first test compound (designated as Compound A) is diluted horizontally in a 96 well microtiter plate in rows B-E using a 3-fold dilution series for 10 dilution points. A second test compound (designated as Compound B) is diluted horizontally in a separate 96 well microtiter plate in rows D-G using a 3-fold dilution series for 10 dilution points. The two compounds are combined using equal volumes from each drug plate into cell culture media. This results in a 1:50 dilution of the drugs in the cell culture media. Compound A is individually titrated in rows B and C, while only Compound B is dosed in rows F and G of the plate. An additional 1:10 dilution of the drugs is performed in cell culture media prior to addition to the cells. Drug addition to the cells results in a further 1:2 dilution of drugs. The total dilution of the drug plate to the cells is 1:1000. The final dosing concentration range for Compound B was 0.008-150.0 nM and was 0.013-250.0 nM for Compound A. The positive control consists of culture media with DMSO at 0.1% and cells and no drug. The negative control consists of culture media with DMSO at 0.1%, solution.

Assays were performed in 96 well microtiter plates with appropriate seeding densities estimated from previous studies of each cell line. Following dosing, the cell lines are incubated at 37° C., 5% CO₂ in humid air for 72 hours. Cell proliferation was measured using the CellTiter Glo (Promega Corporation, Madison, Wis., USA) reagent according to the manufacturer's protocol. The plates are treated with CellTiter Glo solution and are analyzed for RLU (relative light units) using a Molecular Devices SpectraMax M5 (Sunnyvale, Calif., USA) plate reader.

### Data Analysis

Three independent metrics were used to analyze the combinatorial effects on growth inhibition of Compound B and Compound A.
1. Excess over Highest Single Agent (EOHSA)—One standard criterion for measuring drug combinatorial effects is analyzing the effects on cell growth inhibition in absolute terms. In this case, the combination of drugs is compared to the more responsive of the two individual treatments (single agent). For each combination experiment, the percent effect relative to the highest single agent for each dose along the curve is generated. This measure of “Excess of Highest Single Agent (EOHSA)” is one of the criteria used for evaluating synergy of drug combinations. (Borisy A A Elliott P J, Hurst N R, Lee M S, Lehar J, Price E R, Serbedzija G, Zimmermann G R, Foley M A, Stockwell B R, Keith C T. Systematic discovery of multicomponent therapeutics. Proc Natl Acad Sci USA. 2003 Jun. 24; 100(13):7977-82)
2. Bliss synergy—A second criterion often used to determine combination synergy is evaluating the excess inhibition over Bliss independence or “additivity” (Bliss, C.I, Mexico, DF, The Toxicity of Poisons Applied Jointly. Annals of Applied Biology 1939, Vol 26, Issue 3, August 1939). The model assumes a combined response of the two compounds independently using the following:

Score=Eₐ+E_{b}−(Eₐ*E_{b})
Where Eₐ is the effect (or percent inhibition) of compound A and E_{b} is the effect of compound B. The resulting effect of the combination of the two compounds is compared to their predicted additivity by Bliss and a synergy score is generated for each dose along the response curve.
3. Combination Index (CI)— A third criterion for evaluation of synergy is Combination Index (CI) derived from the Chou and Talalay (Chou T C, Talalay P. Quantitative analysis of dose-effect relationships: the combined effects of multiple drugs or enzyme inhibitors. Adv Enzyme Regul. 1984; 22:27-55). The following equation is a model used for compounds that behave with different mechanisms of action (mutually non-exclusive formula).

$$Combination Index = \frac{{D}_{a} in a \text{:} b}{{IC}_{50 ( a )}} + \frac{{D}_{b} in a \text{:} b}{{IC}_{50 ( b )}} + \frac{( {D}_{a} in a \text{:} b ) ( {D}_{b} in a \text{:} b )}{( {IC}_{50 ( a )} ) ( {IC}_{50 ( b )} )}$$

The lower the CI the more synergy the combination potentially has. A CI greater than 1 suggests that the combination being studied may be antagonistic. CI scores are also generated for inhibitory concentrations of 25% (IC₂₅) and 75% (IC₇₅) by replacing the IC₅₀ in the formula above for each compound with the respective inhibitory concentration.

The percent intensity values were used in model 205 of XLfit in Microsoft Excel to calculate gIC₅₀ values using a 4 parameter logistical fit. The midpoint of the growth window (the gIC₅₀) falls half way between the number of cells at the time of compound addition (T=0) and the growth of control cells treated with DMSO at 72 hrs. The number of cells at time zero (T₀) is divided from the intensity value at the bottom of the response curve (Yₘᵢₙ) to generate a measure for cell death (Yₘᵢₙ/T₀). A value below 1 for Yₘᵢₙ/Tₒ indicates stronger potency with the treatment when compared to higher values.

For EOHSA and Bliss, a synergy score must be seen in both technical replications within an experiment to make an appropriate designation (synergy, modest synergy, etc). Each combination experiment contains a replicate for the two compounds as single agents as well as a technical replicate for the combination.

Synergy scores for EOHSA and Bliss, at extremely low concentrations, (e.g. Dose 1, dose 2) are subject to higher variation and generally excluded from the analysis. Conversely, synergy scores at the highest concentrations (Dose 10), far outside of the therapeutic dosing range, are generally excluded from analysis since the effects observed are more susceptible to off-target events.

For EOHSA and Bliss Synergy measures, a score is generated for each dose along the response curve. Scores were categorized as being ‘Antagonistic’ (<−10), ‘Additive’ (−10-10), ‘Modest Synergy’ (10-20) or ‘Synergistic’ (>20). These scores reflect the percentage over the highest agent or percentage greater than Bliss additivity, depending on which model is being interpreted.

For the Combination Index, the lower the CI, the more synergy the combination potentially has. Scores between 0 and 0.7 were considered to be synergistic, while scores between 0.7 and 0.9 were considered to be modest synergy. All other scores did not indicate synergy for the Combination index.

For those cell lines that never reached an inhibitory concentration of 25% for 1 of the compounds in the combination, a CI value cannot be calculated and ‘NA’ was listed for the Cl.

### Cell Line Mutation Data

Mutation data was collated for the status for the KRAS gene. The data source is the cancer cell line mutation screening data published as part of the Catolog of Somatic Mutations in Cancer database (COSMIC) (Bamford S. et al. Br. J. Cancer. 2004. 91:355-58). In order to ensure that the identity of the cell lines used in the proliferation assay matched that in the COSMIC database, a genotype comparison was done between those cell lines in the sensitivity screen and those in COSMIC. Specifically, this entailed:
1. Calculating the genotypes for each cell line using the Affymetrix 500K ‘SNP Chip’ (Affymetrix, Inc., Sunnyvale, Calif.) and the RLMM algorithm (Rabbee & Speed, Bioinformatics, 2006. 22: 7-12).
2. Identifying the genotype matches of each cell line to those pre-calculated for each cell line having mutation profiles in COSMIC.
3. Assigning mutation status for each cell line in based upon the genotype matches.

### Results

A comprehensive categorization of the degree of synergy was done for each cell line treated with the combination of the PI3K inhibitor Compound B and MEK inhibitor Compound A, Cell lines were considered to have synergy when at least one metric was scored as synergistic. Synergy data for Colon, Pancreatic, and Lung cellines is presented in Table 1-4. Data for pancreatic cell line calculations can be seen in Appendix A Tables 7-9.

TABLE 1
Scores Panel of pancreatic, colon and lung cell lines
used in combination studies.

| | | | KRAS |
|---|---|---|---|
| Cell Line | Organ Site | Diagnosis/Histology | mutation status |
| NCI-H747 | Colon | Adenocarcinoma | G13D |
| LS-1034 | Colon | Adenocarcinoma | A146T |
| SW948 | Colon | Adenocarcinoma | Q61L |
| LS-174T | Colon | Adenocarcinoma | G12D |
| SW116 | Colon | Adenocarcinoma | G12A |
| T84 | Colon | Carcinoma | G13D |
| Colo 201 | Colon | Adenocarcinoma | WT |
| SW403 | Colon | Carcinoma | G12V |
| DLD-1 | Colon | Carcinoma | G13D |
| Colo 205 | Colon | Adenocarcinoma | G12V |
| Colo320 HSR | Colon | Adenocarcinoma | WT |
| SW620 | Colon | Adenocarcinoma | G12V |
| NCI-H508 | Colon | Adenocarcinoma | WT |
| Colo 320DM | Colon | Adenocarcinoma | Unavail |
| SW837 | Colon | Adenocarcinoma | G12C |
| KM-12 | Colon | Adenocarcinoma | WT |
| WiDr | Colon | Adenocarcinoma | WT |
| HCT-8 | Colon | ileocecal colorectal | G13D |
| | | adenocarcinoma | |
| RKO | Colon | Carcinoma | WT |
| HT-29 | Colon | Carcinoma | WT |
| SW480 | Colon | Adenocarcinoma | G12V |
| HCT-15 | Colon | Adenocarcinoma | G13D |
| HCT-116 | Colon | Carcinoma | G13D |
| SW48 | Colon | Adenocarcinoma | WT |
| SW1417 | Colon | Adenocarcinoma | WT |
| HCC2998 | Colon | Carcinoma | A146T |
| Calu 6 | Lung | Adenocarcinoma | Q61K |
| SK-MES-1 | Lung | Squamous cell carcinoma | WT |
| A549 | Lung | Alveoloar basal epithelial- | G12S |
| | | squamous | |
| NCI-H2170 | Lung | Squamous cell carcinoma | WT |
| NCI-H2228 | Lung | Adenocarcinoma | WT |
| NCI-H23 | Lung | Adenocarcinoma | WT |
| NCI-H1792 | Lung | Adenocarcinoma | G12C |
| NCI-H358 | Lung | Branchio-alveolar | G12C |
| NCI-H2122 | Lung | Adenocarcinoma | G12C |
| NCI-H520 | Lung | Squamous cell carcinoma | WT |
| NCI-H1299 | Lung | Non-small cell lung cancer | WT |
| NCI-H1563 | Lung | Adenocarcinoma | WT |
| NCI-H460 | Lung | Large cell carcinoma | Q61H |
| NCI-H2030 | Lung | Adenocarcinoma | G12C |
| BxPC3 | Pancreas | Adenocarcinoma | WT |
| SW1990 | Pancreas | Adenocarcinoma | G12D |
| YAPC | Pancreas | Carcinoma | G12V |
| MiaPaCa | Pancreas | Carcinoma | G12C |
| Capan-1 | Pancreas | Adenocarcinoma | G12V |
| HuP-T4 | Pancreas | Carcinoma | G12V |

Table 1 key
Cell Line = Cell line name
Organ Site = Organ from which cells were derived
Diagnosis/Histology = Pathological diagnosis of tissue
KRAS = Mutation status;
WT = Wild Type

TABLE 2
Basic measures and Synergy calls for each of the Colon cell lines.

| | | | Yₘᵢₙ/ | | | Comb |
|---|---|---|---|---|---|---|
| Cell Line | gIC_{50 (nM)} | Yₘᵢₙ | T₀ | EOHSA | BLISS | Index |
| Colo201 | 0.56 | 4.04 | 0.22 | Modest | Additive | Modest |
| | | | | Synergy | | Synergy |
| Colo205 | 0.67 | −0.41 | −0.06 | Modest | Additive | Synergy |
| | | | | Synergy | | |
| Colo320DM | 7.35 | 15.65 | 1.55 | Modest | Modest | N/A |
| | | | | Synergy | Synergy | |
| Colo320HSR | 53.62 | 22.25 | 4.65 | Antag- | Additive | N/A |
| | | | | onism | | |
| DLD1 | 7.77 | 12.21 | 1.71 | Synergy | Synergy | Synergy |
| HCC2998 | 10.78 | 8.78 | 0.97 | Synergy | Synergy | Synergy |
| HCT116 | 7.50 | 6.31 | 1.24 | Synergy | Synergy | Modest |
| | | | | | | Synergy |
| HCT15 | 5.46 | 10.14 | 1.19 | Synergy | Modest | N/A |
| | | | | | Synergy | |
| HCT8 | 23.70 | 5.64 | 0.76 | Synergy | Modest | Synergy |
| | | | | | Synergy | |
| HT29 | 0.59 | 1.70 | 0.19 | Synergy | Modest | Synergy |
| | | | | | Synergy | |
| KM12 | 21.95 | 8.21 | 0.61 | Synergy | Synergy | Synergy |
| LS1034 | 12.26 | 7.20 | 0.35 | Modest | Antagonism | Modest |
| | | | | Synergy | | Synergy |
| LS174T | 46.60 | 4.02 | 0.19 | Modest | Antagonism | N/A |
| | | | | Synergy | | |
| NCIH508 | 0.08 | 1.90 | 0.07 | Modest | Additive | Synergy |
| | | | | Synergy | | |
| NCIH747 | 2.59 | 4.65 | 0.14 | Modest | Additive | Synergy |
| | | | | Synergy | | |
| RKO | 25.52 | 1.42 | 0.15 | Synergy | Synergy | Synergy |
| SW116 | #N/A | 11.25 | 0.18 | Synergy | Synergy | Synergy |
| SW1417 | 1.09 | 21.32 | 1.21 | Synergy | Additive | Synergy |
| SW403 | 1.04 | 1.51 | 0.05 | Synergy | Additive | Synergy |
| SW48 | 0.68 | −0.30 | −0.02 | Synergy | Modest | Synergy |
| | | | | | Synergy | |
| SW480 | 1.35 | 17.03 | 0.67 | Synergy | Additive | Synergy |
| SW620 | 6.95 | 14.69 | 2.12 | Synergy | Synergy | N/A |
| SW837 | 1.24 | 13.39 | 0.46 | Synergy | Additive | Synergy |
| SW948 | 32.97 | 2.82 | 0.14 | Synergy | Synergy | Synergy |
| T84 | 0.78 | −3.52 | −0.12 | Synergy | Synergy | Synergy |
| WiDr | 6.34 | −0.02 | 0.00 | Modest | Additive | Modest |
| | | | | Synergy | | Synergy |

Tables 2-7 Key:
Cell Line = Tumor-derived cell line
gIC₅₀ = Concentration of compound (nM) required to cause 50% growth inhibition
Yₘᵢₙ = The minimum cellular growth in the presence of Compound B (relative to DMSO control) as measured by % of that at T = 0 (number of cells at time of Compound B addition). A negative number indicates a net loss of cells relative to that at T = 0.
Yₘᵢₙ/T₀ = Yₘᵢₙ value divided by the T0 value whereas the Yₘᵢₙ is derived from the concentration-response curve and the T0 value represents the number of cells at the time of compound addition (CTG measurement).
EOHSA = Excess over highest single agent determination
BLISS = Bliss synergy determination
Comb Index = Combination Index score

TABLE 3
Basic measures and Synergy calls for each of the Lung cell lines.

| Cell | | | Yₘᵢₙ/ | | | Comb |
|---|---|---|---|---|---|---|
| Line | gIC_{50 (nM)} | Yₘᵢₙ | T₀ | EOHSA | BLISS | Index |
| NCIH2122 | 4.01 | 1.34 | 0.08 | Antagonism | Antag- | Synergy |
| | | | | | onism | |
| A549 | 3.00 | 0.32 | 0.06 | Synergy | Modest | Synergy |
| | | | | | Synergy | |
| Calu6 | 2.79 | 3.51 | 0.22 | Synergy | Synergy | Synergy |
| NCIH1299 | 1.55 | 11.11 | 1.14 | Synergy | Modest | Synergy |
| | | | | | Synergy | |
| NCIH1563 | 0.41 | 1.56 | 0.04 | Synergy | Synergy | Synergy |
| NCIH1792 | 2.89 | 1.34 | 0.05 | Synergy | Modest | Synergy |
| | | | | | Synergy | |
| NCIH2030 | 0.92 | 8.19 | 0.43 | Synergy | Modest | Synergy |
| | | | | | Synergy | |
| NCIH2170 | 3.25 | 4.05 | 0.22 | Synergy | Modest | Synergy |
| | | | | | Synergy | |
| NCIH2228 | 2.95 | 3.29 | 0.28 | Synergy | Synergy | Synergy |
| NCIH23 | 1.11 | 7.72 | 0.47 | Synergy | Synergy | Synergy |
| NCIH358 | 4.97 | 2.40 | 0.11 | Synergy | Synergy | Synergy |
| NCIH460 | 1.91 | 5.11 | 1.25 | Synergy | Modest | Synergy |
| | | | | | Synergy | |
| NCIH520 | 5.97 | 15.31 | 1.13 | Synergy | Synergy | Synergy |
| SKMES1 | 2.91 | 0.09 | 0.00 | Synergy | Synergy | Synergy |

TABLE 4
Basic measures and Synergy calls for each of the Pancreatic cell lines.

| Cell | | | | | | Comb |
|---|---|---|---|---|---|---|
| Line | gIC_{50 (nM)} | Yₘᵢₙ | Yₘᵢₙ/T₀ | EOHSA | BLISS | Index |
| BxPC3 | 0.43 | 0.81 | 0.02 | Synergy | Modest | Synergy |
| | | | | | Synergy | |
| Capan1 | 1.17 | 12.34 | 0.37 | Synergy | Modest | Synergy |
| | | | | | Synergy | |
| HUPT4 | 0.22 | 3.77 | 0.12 | Synergy | Synergy | Synergy |
| MiaPaCa | 5.10 | 8.10 | 1.03 | Synergy | Modest | Synergy |
| | | | | | Synergy | |
| SW1990 | 5.28 | 3.28 | 0.21 | Synergy | Synergy | Synergy |
| YAPC | 7.34 | 16.35 | 0.81 | Synergy | Modest | Synergy |
| | | | | | Synergy | |

### Study #2. Breast Cancer Cell Lines Analyzed for Estrogen Receptor

### Experimental Preparation(s)

Combination drug tests with the MEK inhibitor (Compound A) and the PI3K inhibitor (Compound B) were conducted using a panel of cell lines from human breast cancers (n=10)(Table 1). Cell lines were purchased commercially [from ATCC (Manassas, Va., USA) or DSMZ (Braunschweig, Germany)] and grown in RPMI-1640 supplemented with 2 mM glutamine, 1 mM sodium pyruvate and 10% fetal bovine serum and maintained at 37° C. and 5% CO₂ in a humid incubator.

### Experimental Protocol(s)

### Fixed Ratio Drug Combination Assay

The dilution design of the Fixed Ratio Drug Combination Assay can be seen in FIG. 1. First, the test compounds were prepared as 10 mM stocks in 100% dimethyl sulfoxide (DMSO). Further dilutions of the compounds were made with DMSO. The first test compound (designated as Compound 1) is diluted horizontally in a 96 well microtiter plate in rows B-E using a 3-fold dilution series for 10 dilution points. A second test compound (designated as Compound 2) is diluted horizontally in a separate 96 well microtiter plate in rows D-G using a 3-fold dilution series for 10 dilution points. The two compounds are combined using equal volumes from each drug plate into cell culture media. This results in a 1:50 dilution of the drugs in the cell culture media. Compound 1 is individually titrated in rows B and C, while only Compound 2 is dosed in rows F and G of the plate. An additional 1:10 dilution of the drugs is performed in cell culture media prior to addition to the cells. Drug addition to the cells results in a further 1:2 dilution of drugs. The total dilution of the drug plate to the cells is 1:1000. The final dosing concentration range for GSK2126458A was 0.008-150.0 nM and was 0.013-250.0 nM for GSK1120212B. The positive control consists of culture media with DMSO at 0.1% and cells and no drug. The negative control consists of culture media with DMSO at 0.1%, solution.

Assays were performed in 96 well microtiter plates with appropriate seeding densities estimated from previous studies of each cell line. Following dosing, the cell lines are incubated at 37° C., 5% CO₂ in humid air for 72 hours. Cell proliferation was measured using the CellTiter Glo (Promega Corporation, Madison, Wis., USA) reagent according to the manufacturer's protocol. The plates are treated with CellTiter Glo solution and are analyzed for RLU (relative light units) using a Molecular Devices SpectraMax M5 (Sunnyvale, Calif., USA) plate reader.

### Data Analysis

The percent intensity values were used in model 205 of XLfit in Microsoft Excel to using a 4 parameter logistical fit to calculate response metrics, including the midpoint of the growth window gIC₅₀, number of cells at time zero (Tₒ), and the intensity value at the bottom of the response curve Yₘᵢₙ Each combination experiment contains a replicate for the two compounds as single agents as well as a technical replicate for the combination. Average values were used for subsequent analysis.

Three independent metrics were used to analyze the combinatorial effects on growth inhibition of Compound A and Compound B. These include i.) Excess over Highest Single Agent (EOHSA; Borisy et al, 2003; FDA 21 CFR 300.50), ii.) Bliss synergy and iii.) Combination Index (CI). Descriptions of these three metrics and methods for their calculation are described above. Also, criteria used to determine the degree of synergy by each metric is also found above. For EOHSA and Bliss, a synergy score must be seen in both technical replications within an experiment to make an appropriate designation (synergy, modest synergy, etc). Briefly, a cell line was considered synergistic when at least one of the three metrics (CI, Bliss Synergy, EOHSA) scored in the synergistic range as stated above.

Estrogen Receptor (ER) and Progesterone receptor (PR) transcript abundance was measured for all cell lines using the Affymetrix U133 Plus2 GeneChips in triplicate. Transcript abundance was estimated by normalizing all probe signal intensities were normalized to a value of 150 using the mass algorithm in the Affymetrix Microarray Analysis Suite 5.0. For subsequent analysis, a representative probe was chosen and the average probe intensity was used for triplicates.

### Results

A comprehensive categorization of the degree of synergy was done for each cell line treated with the combination Compounds A and B.

TABLE 5
Scores Panel of breast cancer cell lines used
in combination studies.

| Cell Line | Organ Site | Diagnosis/Histology |
|---|---|---|
| DU4475 | Breast | Carcinoma |
| EFM19 | Breast | Carcinoma |
| HCC1954 | Breast | Carcinoma |
| HCC70 | Breast | Carcinoma |
| MT3 | Breast | Carcinoma |
| MX1 | Breast | Carcinoma |
| NCI-ADR-RES | Breast | Carcinoma |
| UACC893 | Breast | Carcinoma |
| T47D | Breast | Carcinoma |
| ZR-75-1 | Breast | Carcinoma |

TABLE 6
Basic measures and Synergy calls for each of the breast cancer cell lines.

| | | | Yₘᵢₙ/ | | | Comb |
|---|---|---|---|---|---|---|
| Cell Line | gIC_{50 (nM)} | Yₘᵢₙ | T₀ | EOHSA | BLISS | Index |
| DU4475 | 0.12 | −0.29 | −0.02 | No | No Synergy | Modest |
| | | | | Synergy | | Synergy |
| EFM19 | 3.43 | 14.97 | 0.49 | No | No Synergy | N/A |
| | | | | Synergy | | |
| HCC1954 | 6.53 | 0.32 | 0.03 | Synergy | Synergy | Synergy |
| HCC70 | 0.31 | −0.63 | −0.02 | Synergy | Synergy | Synergy |
| MT3 | 4.47 | 5.59 | 0.39 | Synergy | Synergy | Synergy |
| MX1 | 5.99 | 13.99 | 1.04 | No | No Synergy | N/A |
| | | | | Synergy | | |
| NCI-ADR- | 49.01 | 36.28 | 2.17 | Synergy | Synergy | N/A |
| RES | | | | | | |
| UACC893 | 3.44 | −0.32 | −0.01 | Modest | Modest | N/A |
| | | | | Synergy | Synergy | |
| T47D | 0.93 | 23.68 | 0.97 | Synergy | Synergy | N/A |
| ZR-75-1 | 0.03 | 2.73 | 0.06 | No | No Synergy | Synergy |
| | | | | Synergy | | |

TABLE 7
Panel of breast cell lines (n = 10), ER/PR transcript abundance
measurements used in combination experiments for Compound B
and Compound A.

| | | Progesterone |
|---|---|---|
| | Estrogen Receptor | Receptor |
| CL Name | Expression (mas5) | Expression (mas5) |
| HCC70 | 75 | 33 |
| DU4475 | 32 | 30 |
| HCC1954 | 128 | 34 |
| NCI-ADR-RES | 42 | 120 |
| UACC893 | 114 | 37 |
| EFM19 | 2122 | 1333 |
| MT3 | 34 | 25 |
| MX1 | 1318 | 74 |
| T47D | 1329 | 3621 |
| ZR-75-1 | 822 | 1103 |

### Study #3. In Vitro Cell Growth Inhibition and Apoptosis Induction by Compounds A & B in a Panel Hepatocellular Carcinoma Cell Lines and a Panel Breast Cancer Cell Lines Analyzed for Her2 DNA Copy Number Changes

### Cell Lines and Growth Conditions

Human tumor cell lines from hepatocellular carcinoma (HCC), C3A, Hep3B, HepG2, PLC/PRF/5, SNU182, SNU387, SNU398, SNU423, SNU449 and SNU475 were purchased from the ATCC. Human breast tumor cell lines, HCC2218, HCC1419, BT-474, SK-BR-3, UACC893, JIMT-1, MDA-MB-361, HCC202, MDA-MB-175-VII, HCC1569, HCC1937, HCC38, MDA-MB-157, HCC1954, HCC1500, BT483, KPL-1, SUM225 and ZR-75-1 from ATCC, SUM52 and SUM190 from Asterand, PLC (Detroit Mich.), were cultured in RPMI 1640 medium containing 10% FBS; SKBR3-W13 and BT-474-J4 cultured in RPMI 1640 medium containing 10% FBS and 1 μM lapatinib; KPL4 line was kindly provided by Dr Junichi Kurebayashi (Kawasaki Medical School, Okayama, Japan) and cultured in DMEM containing 5% FBS. JIMT-1 from European Collection of Cell Cultures (UK), is a line derived from a patient clinically resistant to trastuzumab (Herceptin®). SK-BR-3-W13 is a single cell clone isolated by a cloning cylinder after a single treatment of SK-BR-3 cells with 0.5 μM lapatinib. BT-474-J4 is a single cell clone derived from a pool of BT-474 cells that were selected to grow in lapatinib to a concentration of 3 μM.

### Cell Growth Inhibition Assay and Combination Data Analysis

Cells were seeded in a 96-well tissue culture plate (NUNC 136102) of RPMI medium containing 10% FBS at 500-2,000 cells per well. Approximately 24 hours after plating, cells were exposed to ten, two-fold or three-fold serial dilutions of either Compound A or B or the combination of the two agents at a 2:1 molar ratio (Compounds A and B respectively). In some cases, cells were grown in RPMI media containing 10% FBS and in the presence or absence of 2 ng/mL hepatocyte growth factor (HGF). Cells were incubated in the presence of compounds for 3 days. ATP levels were determined by adding Cell Titer Glo® (Promega) according to the manufacturer's protocol. Briefly, Cell Titer Glo® was added to each plate, incubated for 20 minutes then luminescent signal was read on the SpectraMax L plate reader with a 0.5 sec integration time. All assays were run at least in duplicate.

Inhibition of cell growth was estimated after treatment with compound or combination of compounds for three days and comparing the signal to cells treated with vehicle (DMSO). Cell growth was calculated relative to vehicle (DMSO) treated control wells. Concentration of compound that inhibits 50% of control cell growth (IC₅₀) was back-interpolated when y=50% of DMSO treated control wells using nonlinear regression with the equation:

$$y = \frac{A + ( B - A )}{1 + {( \frac{C}{x} )}^{D}}$$

where A is the minimum response (yₘᵢₙ), B is the maximum response (Yₘₐₓ), C is the inflection point of the curve (EC₅₀) and D is the Hill coefficient.

Combination effects on potency were evaluated using the Combination Index (CI) and Excess Over Highest Single Agent (EHOSA) methods.

In this study, co-administration of Compounds A & B exhibit a synergistic interaction in a specific cell line to potency or on the response scale, if the CI<0.9 or the EOHSATD>0.

### Cell Apoptosis Assays—Caspase-3/7 Activation and DNA Fragmentation

For investigation of the induction of apoptosis, all cell lines were plated at 5,000 cells per well in a 96-well tissue culture plate and allowed to attach for approximately 24 hours. Cells were then treated with compounds as described above. 24 hours after compound treatment, the levels of active caspase 3 and caspase 7 were determined with the Caspase Glo™ 3/7 (Promega, cat G8093) according to the instructions provided by the manufacturer. 48 hours after treatment with compound, levels of apoptosis were estimated using the Roche Cell Death ELISA (Roche, Inc., Basel, Switzerland; Cat. No. 11 774 425 001) following the instructions provided by the manufacturer.

For the purposes of molecular characterization of selected cell lines, the expression levels of several key proteins were measured by western blot. These included E-cadherin (CDH-1), vimentin (VIM), HER3STAT3, MET, AKT and ERK1/2. Actin was used as a control in each case

### DNA Copy Number

DNA Copy number data on the HER2 gene was collected for all breast cancer cell lines using the Affymetrix 500K chip (Affymetrix Inc, Sunnyvale, Calif.). Briefly DNA was extracted from each line, digested with the restriction enzyme Nsp or Sty, ligated to an adaptor and amplified by PCR. After PCR, DNA was fragmented, labeled, denatured, and hybridized to the Affymetrix 500K chip. Upon completion of hybridization, each assay was washed and stained. Image data were acquired. Similarly collected data from a panel 10 diploid non-tumorigenic lymphoblastic cell lines were used to calculate DNA copy number. All ‘SNP Chip’ images ('CEL files'), were extracted, read and normalized using the dChip software package (Lin et al. 2004. Bioinformatics. 20:1233-40). SNP-wise ‘copy-number ratios’ (log₂ scale) were calculated for all cancer cell lines using the lymphoblastic reference panel and analyzed by circular binary segmentation to reduce noise (Olshen et al. 2004. Biostatistics. 5:557-72). Cell lines with log₂ ratios of HER2>0.65 were considered HER2+.

### Results

### Effects of Cell Growth Inhibition and Apoptosis on Hepatocellular Carcinoma Cell Lines by Compound A and Compound B Combination

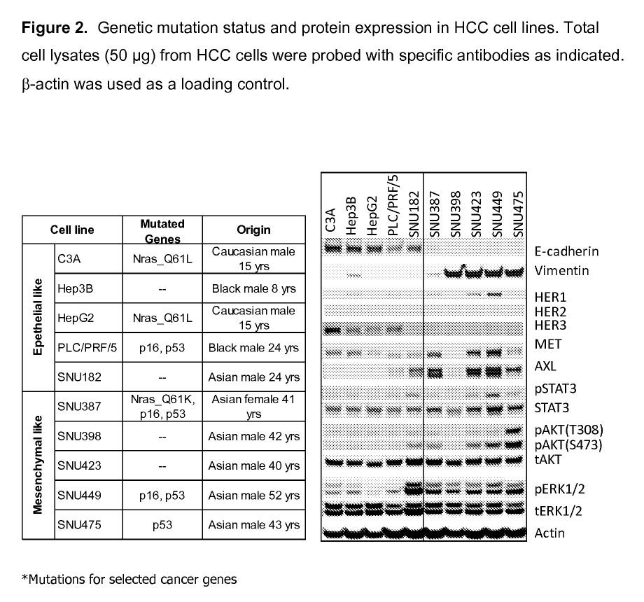

The genetic backgrounds and protein expression analyzed by Western blot in 10 hepatocellular carcinoma (HCC) cell lines were shown in FIG. 2. The cell lines C3A, Hep3B, HepG2, PLC/PRF/5 and SNU182 express high levels of CDH-1 and extremely low to low levels of VIM, whereas SNU387, SNU398, SNU423, SNU449 and SNU475 cell lines express relatively high levels of VIM and extremely low levels of CDH-1. High levels of CDH-1 and low or no VIM is characteristic of epithelial cells, while high VIM and low CDH-1 is characteristic of mesenchymal cells. Therefore, C3A, Hep3B, HepG2, PLC/PRF/5 and SNU182 are defined as epithelial-like and SNU387, SNU398, SNU423, SNU449 and SNU475 as mesenchymal-like cells. This is consistent with the fact that HER3 is highly expressed in the epithelial-like HCC lines and AXL is highly expressed in mesenchymal-like cells (data also shown in FIG. 2). STAT3, AKT and ERK1/2 (total protein) were expressed at a similar level in epithelial-like and mesenchymal-like cell lines, while MET expression was variable, but not differentially-associated with either group of cells. Phosphorylation/activation of AKT is preferentially observed in mesenchymal-like cell lines, with higher levels of pAKT-S473 than pAKT-T308. pERK1/2 was also differentially, but not exclusively, present in mesenchymal-like cells.

The effects of cell growth inhibition by Compound A, Compound B and their combination were determined in 10 HCC cell lines. The mean IC₅₀s (from at least two independent experiments) and the combination effects at IC₅₀s are summarized in Table 8. Three epithelial-like HCC cell lines (HepG2, C3A and Hep3B) were strongly sensitive to cell growth inhibition by Compound A (IC₅₀<37 nM), and SNU 182 and PLC/PRF/5 epithelial-like cell lines were weakly sensitive to Compound A (IC₅₀=1.2-2.8 μM). Two mesenchymal-like HCC cell lines (SNU387 and SNU423) were moderately sensitive to cell growth inhibition by Compound A (IC₅₀=74-577 nM) while three mesenchymal-like cell lines (SNU398, SNU449 and SNU475) were not sensitive to cell growth inhibition by Compound A. All 10 HCC lines were sensitive to cell growth inhibition by Compound B (IC₅₀<103 nM). Furthermore, combination treatment with Compound A and Compound B (1:2 ratio) showed strong synergy as demonstrated by the combination index values ranging from 0.22 to 0.78 or greater than the best single agent by EOHSATD analysis (5-20 ppt) and EOHSA analysis (12-27 ppt) in 8 of 10 HCC cell lines. The presence of HGF had no consistent effect on responsiveness to either drug alone or in combination.

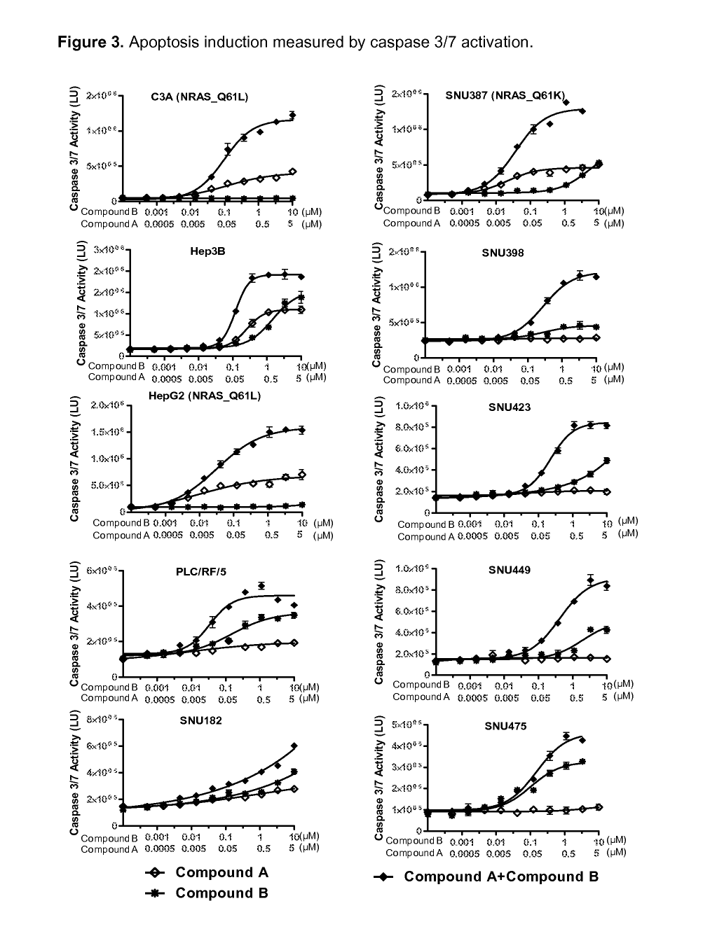

These 10 HCC lines were further evaluated for the ability of Compound A, Compound B or the combination of Compound A and Compound B to induce apoptosis as determined by caspase 3/7 activities. Activation of caspase 3 is a hallmark of induction of apoptosis. Representative caspase 3/7 activity curves for these cells are provided in FIG. 3. All cell lines except SNU182 showed strong enhancement of apoptosis by combination treatment with Compound A and Compound B relative to single agent treatment with Compound A or Compound B. SNU182 cells showed moderate enhancement of apoptosis by combination treatment with Compound A and Compound B relative to their single agent treatment.

### Effects of Cell Growth Inhibition on Human Breast Tumor Cell Lines Measured for Her2 Levels by Compound A and Compound B Combination

Analysis of copy number alterations in the HER2 gene identified 14 as HER2 positive (HER2+) breast tumor lines. These were BT474, BT474-J4, HCC1419, HCC1954, HCC202, HCC2218, JIMT-1, KPL-4, MDA-MB-361, SK-BR-3, SK-BR-3-W13, SUM190, SUM225 and UACC893. A total of 10 were considered HER2 negative (HER2—). These include BT483, HCC1500, HCC1569, HCC1937, HCC38, KPL-1, MDA-MB-157, MDA-MB-175-VII, ZR-75-1 and SUM52.

The effects of cell growth inhibition by Compound A, Compound B and their combination were determined these 25 cell lines, The mean IC₅₀ values (from at least two independent experiments) and the combination effects at IC₅₀ values are summarized in Table 9.

Cell lines SUM52 and MDA-MB-17511 are sensitive to Compound A with IC₅₀ values of less than or equal to 0.099 μM. In contrast, all lines except HCC1937, SK-Br-3-W13 and MDA-MB-157 are sensitive to Compound B with IC₅₀<0.1 μM. The combination of Compound A and Compound B showed synergy with combination index (CI) values between 0.48 and 0.83 and greater than the most active single agent analysis (EOSHA) between 15 and 25 ppts in SUM52, HCC1954 (HER2+) and MDA-MB-17511 (HER2—) cell lines. The combination of Compound A and Compound B also showed a benefit of greater than the most active single agent analysis (EOSHA) between 10 and 15 ppts in a subset of HER2+(SUM190, HCC202) and HER2— lines (MDA-MB-157, HCC1937). The combination of Compound A and Compound B showed a comparable effect to the most active single agent in the rest of the lines. The mean EOHSA score for Her2+lines (n=14) was 9.1 (±7.4), while the mean score for the Her2-line s (n=10) was 6.9 (±7.2). These EOHSA scores did not significantly differ between groups (p=0.45; t-test).

TABLE 8
Cell growth inhibition by Compound A, Compound B and their combination in
human hepatocellular carcinoma tumor cell lines.

| | | | Single agent (IC₅₀, μM) | | Combination (IC₅₀, μM) | | Combination Effect (A:B = 1:2) | | |
|---|---|---|---|---|---|---|---|---|---|
| | | HGF | Compound | | Compound | | | EOHSATD, | EOHSA, |
| Cell line | | (2 ng/ml) | A | Compound B | A | Compound B | CI | ppt | ppt |
| Epethelial like | HepG2 | − | 0.001 ± 0.000 | 0.009 ± 0.008 | 0.001 ± 0.000 | 0.001 ± 0.001 | 0.72 ± 0.05 | <0 | 13.5 ± 5.0 |
| | | + | 0.003 ± 0.001 | 0.009 ± 0.005 | 0.001 ± 0.000 | 0.002 ± 0.001 | 0.57 ± 0.12 | 8.2 ± 1.1 | 22.7 ± 7.3 |
| | C3A | − | 0.010 ± 0.003 | 0.013 ± 0.001 | 0.001 ± 0.000 | 0.002 ± 0.000 | 0.30 ± 0.06 | 11.4 ± 3.6 | 20.6 ± 1.4 |
| | | + | 0.036 ± 0.011 | 0.013 ± 0.001 | 0.002 ± 0.000 | 0.004 ± 0.001 | 0.39 ± 0.03 | 11.1 ± 2.2 | 16.5 ± 2.1 |
| | Hep3B | − | 0.026 ± 0.008 | 0.028 ± 0.002 | 0.005 ± 0.003 | 0.010 ± 0.005 | 0.61 ± 0.26 | 12.6 ± 9.9 | 19.2 ± 9.9 |
| | | + | 0.037 ± 0.008 | 0.057 ± 0.020 | 0.006 ± 0.000 | 0.012 ± 0.001 | 0.44 ± 0.18 | 19.3 ± 10.2 | 25.0 ± 9.7 |
| | SNU182 | − | 1.758 ± 0.224 | 0.017 ± 0.001 | 0.003 ± 0.000 | 0.005 ± 0.000 | 0.31 ± 0.01 | 13.4 ± 2.0 | 20.5 ± 2.7 |
| | | + | 1.282 ± 1.223 | 0.017 ± 0.004 | 0.002 ± 0.000 | 0.005 ± 0.000 | 0.29 ± 0.09 | 14.9 ± 6.0 | 21.5 ± 6.2 |
| | PLC/RF/5 | − | 1.604 ± 0.509 | 0.018 ± 0.012 | 0.002 ± 0.001 | 0.005 ± 0.002 | 0.32 ± 0.12 | 15.4 ± 10.6 | 22.2 ± 10.8 |
| | | + | 2.871 ± 1.556 | 0.015 ± 0.001 | 0.003 ± 0.001 | 0.006 ± 0.001 | 0.41 ± 0.06 | 8.4 ± 1.8 | 15.3 ± 1.4 |
| Mesenchymal | SNU423 | − | 0.218 ± 0.130 | 0.023 ± 0.016 | 0.006 ± 0.003 | 0.011 ± 0.007 | 0.55 ± 0.05 | 4.8 ± 1.8 | 11.7 ± 2.1 |
| like | | + | 0.577 ± 0.569 | 0.021 ± 0.007 | 0.008 ± 0.004 | 0.016 ± 0.008 | 0.78 ± 0.09 | <0 | 5.9 ± 2.7 |
| | SNU449 | − | >5 | 0.024 ± 0.005 | 0.013 ± 0.007 | 0.026 ± 0.013 | NA | <0 | −0.6 ± 5.5 |
| | | + | >5 | 0.024 ± 0.011 | 0.015 ± 0.003 | 0.029 ± 0.006 | NA | <0 | −3.5 ± 4.3 |
| | SNU475 | − | >5 | 0.050 ± 0.019 | 0.016 ± 0.002 | 0.033 ± 0.003 | NA | <0 | 9.0 ± 11.4 |
| | | + | >5 | 0.039 ± 0.017 | 0.018 ± 0.003 | 0.036 ± 0.007 | NA | <0 | 0.3 ± 16.9 |
| | SNU398 | − | >5 | 0.098 ± 0.021 | 0.009 ± 0.003 | 0.020 ± 0.007 | NA | 20.1 ± 4.8 | 26.9 ± 5.7 |
| | | + | >5 | 0.083 ± 0.002 | 0.008 ± 0.001 | 0.016 ± 0.001 | NA | 20.1 ± 1.7 | 26.8 ± 2.0 |
| | SNU387 | − | 0.074 ± 0.046 | 0.103 ± 0.010 | 0.006 ± 0.002 | 0.012 ± 0.004 | 0.22 ± 0.00 | 14.2 ± 4.9 | 21.0 ± 0.8 |
| | | + | 0.094 ± 0.021 | 0.071 ± 0.010 | 0.008 ± 0.003 | 0.016 ± 0.005 | 0.34 ± 0.13 | 11.3 ± 4.9 | 15.4 ± 4.8 |

Table 8 Key:
HGF: Hepatocyte Growth Factor;
‘+’ = in the presence,
‘−’ = in the absence.
IC₅₀: the concentration of Compound(s) that reduces cell growth by 50%;
CI; Combination Index;
NA = not applicable
EOHSATD: Excess Over Highest Single Agent at Total Dose, measured as a percentage
EOHSA: Excess over Highest Single Agent, measured as a percentage

TABLE 9
Cell growth inhibition by Compound A, Compound B and their combination
in breast tumor cell lines.

| | | | | Combination (A:B = 1:1) | | |
|---|---|---|---|---|---|---|
| | | Single agent (IC₅₀, μM) | | Compound | | |
| Breast cell | | | Compound | A or B (IC₅₀, | | EOHSA |
| lines | HER2 | Compound A | B | μM)* | CI | (AorB; ppt) |
| HCC1954 | HER2+ | 1.018 ± 0.839 | 0.011 ± 0.003 | 0.005 ± 0.002 | 0.48 ± 0.07 | 24.5 ± 3.3 |
| SUM190 | HER2+ | >1 | 0.006 ± 0.003 | 0.003 ± 0.001 | NA | 15.0 ± 5.6 |
| HCC202 | HER2+ | >1 | 0.045 ± 0.004 | 0.013 ± 0.000 | NA | 11.5 ± 0.3 |
| HCC2218 | HER2+ | >1 | 0.018 ± 0.013 | 0.009 ± 0.002 | NA | 9.6 ± 9.4 |
| JIMT-1 | HER2+ | >1 | 0.028 ± 0.005 | 0.018 ± 0.002 | NA | 9.3 ± 6.7 |
| UACC893 | HER2+ | >1 | 0.003 ± 0.003 | 0.002 ± 0.002 | NA | 8.6 ± 3.8 |
| SK-BR-3-W13 | HER2+ | >1 | 0.129 ± 0.099 | 0.091 ± 0.107 | NA | 6.8 ± 5.2 |
| BT474-J4 | HER2+ | >1 | 0.014 ± 0.008 | 0.012 ± 0.011 | NA | 5.6 ± 4.9 |
| MDA-MB-361 | HER2+ | >1 | 0.009 ± 0.002 | 0.007 ± 0.002 | NA | 4.3 ± 4.8 |
| HCC1419 | HER2+ | >1 | 0.024 ± 0.014 | 0.020 ± 0.011 | NA | 3.7 ± 3.1 |
| KPL4 | HER2+ | >1 | 0.003 ± 0.001 | 0.003 ± 0.001 | NA | −1.7 ± 7.8 |
| SK-BR-3 | HER2+ | >1 | 0.024 ± 0.020 | 0.024 ± 0.016 | NA | −1.6 ± 2.4 |
| SUM225 | HER2+ | >1 | 0.013 ± 0.014 | 0.011 ± 0.011 | NA | 1.2 ± 2.7 |
| BT474 | HER2+ | >1 | 0.030 ± 0.007 | 0.033 ± 0.010 | NA | −0.9 ± 1.1 |
| SUM52 | HER2− | 0.009 ± 0.005 | 0.004 ± 0.000 | 0.002 ± 0.000 | 0.83 ± 0.11 | 22.7 ± 5.5 |
| MDA-MB-175- | HER2− | 0.099 ± 0.097 | 0.007 ± 0.001 | 0.004 ± 0.001 | 0.60 ± 0.08 | 15.0 ± 5.1 |
| VII | | | | | | |
| MDA-MB-157 | HER2− | >1 | >1 | 0.077 ± 0.047 | NA | 15.5 ± 4.2 |
| HCC1937 | HER2− | >1 | 0.114 ± 0.063 | 0.069 ± 0.048 | NA | 11.0 ± 3.8 |
| KPL1 | HER2− | >1 | 0.008 ± 0.004 | 0.007 ± 0.002 | NA | 2.0 ± 3.4 |
| ZR-75-1 | HER2− | >1 | 0.006 ± 0.001 | 0.005 ± 0.001 | NA | 3.2 ± 0.5 |
| BT483 | HER2− | >1 | 0.106 ± 0.081 | 0.082 ± 0.083 | NA | 4.4 ± 4.4 |
| HCC1500 | HER2− | >1 | 0.061 ± 0.036 | 0.035 ± 0.012 | NA | 9.5 ± 5.8 |
| HCC38 | HER2− | >1 | 0.095 ± 0.034 | 0.059 ± 0.005 | NA | 9.7 ± 7.8 |
| HCC1569 | HER2− | >1 | 0.076 ± 0.042 | 0.095 ± 0.082 | NA | −1.6 ± 7.5 |

Table 9 Key:
HER2: HER2+ = HER2 positive, log2 ratios of HER2 DNA copy number >0.65; HER2− = HER2 negative, log2 ratios of HER2 DNA copy number <0.65.
*IC₅₀: the concentration of Compound A in the presence of equal molar Compound B that reduces cell growth by 50%;
CI; Combination Index;
NA = not applicable;
EOHSA: Excess over Highest Single Agent, measured as a percentage.

### Example 1

### Capsule Composition

An oral dosage form for administering a combination of the present invention is produced by filing a standard two piece hard gelatin capsule with the ingredients in the proportions shown in Table I, below.

TABLE I

| INGREDIENTS | AMOUNTS |
|---|---|
| N-{3-[3-cyclopropyl-5-[(2-fluoro-4-iodophenyl)amino]-6,8- | 5 mg |
| dimethyl-2,4,7-trioxo-3,4,6,7-tetrahydropyrido[4,3- | |
| d]pyrimidin-1(2H)-yl]phenyl}acetamide dimethyl sulfoxide | |
| (the dimethyl sulfoxide solvate of Compound A) | |
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Mannitol | 50 mg |
| Talc | 25 mg |
| Magnesium Stearate | 2 mg |

### Example 2

### Capsule Composition

An oral dosage form for administering one of the compounds of the present invention is produced by filing a standard two piece hard gelatin capsule with the ingredients in the proportions shown in Table II, below.

TABLE II

| INGREDIENTS | AMOUNTS |
|---|---|
| N-{3-[3-cyclopropyl-5-[(2-fluoro-4-iodophenyl)amino]-6,8- | 5 mg |
| dimethyl-2,4,7-trioxo-3,4,6,7-tetrahydropyrido[4,3- | |
| d]pyrimidin-1(2H)-yl]phenyl}acetamide dimethyl sulfoxide | |
| (the dimethyl sulfoxide solvate of Compound A) | |
| Mannitol | 55 mg |
| Talc | 16 mg |
| Magnesium Stearate | 4 mg |

### Example 3

### Capsule Composition

An oral dosage form for administering one of the compounds of the present invention is produced by filing a standard two piece hard gelatin capsule with the ingredients in the proportions shown in Table III, below.

TABLE III

| INGREDIENTS | AMOUNTS |
|---|---|
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Mannitol | 50 mg |
| Talc | 25 mg |
| Magnesium Stearate | 2 mg |

### Example 4

### Tablet Composition

The sucrose, microcrystalline cellulose and the compounds of the invented combination, as shown in Table IV below, are mixed and granulated in the proportions shown with a 10% gelatin solution. The wet granules are screened, dried, mixed with the starch, talc and stearic acid, then screened and compressed into a tablet.

TABLE IV

| INGREDIENTS | AMOUNTS |
|---|---|
| N-{3-[3-cyclopropyl-5-[(2-fluoro-4-iodophenyl)amino]-6,8- | 5 mg |
| dimethyl-2,4,7-trioxo-3,4,6,7-tetrahydropyrido[4,3- | |
| d]pyrimidin-1(2H)-yl]phenyl}acetamide dimethyl sulfoxide | |
| (the dimethyl sulfoxide solvate of Compound A) | |
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Microcrystalline cellulose | 60 mg |
| sucrose | 5 mg |
| starch | 10 mg |
| talc | 5 mg |
| stearic acid | 2 mg |

### Example 5

### Tablet Composition

The sucrose, microcrystalline cellulose and one of the compounds of the invented combination, as shown in Table V below, are mixed and granulated in the proportions shown with a 10% gelatin solution. The wet granules are screened, dried, mixed with the starch, talc and stearic acid, then screened and compressed into a tablet.

TABLE V

| INGREDIENTS | AMOUNTS |
|---|---|
| N-{3-[3-cyclopropyl-5-[(2-fluoro-4-iodophenyl)amino]-6,8- | 5 mg |
| dimethyl-2,4,7-trioxo-3,4,6,7-tetrahydropyrido[4,3- | |
| d]pyrimidin-1(2H)-yl]phenyl}acetamide dimethyl sulfoxide | |
| (the dimethyl sulfoxide solvate of Compound A) | |
| Microcrystalline cellulose | 30 mg |
| sucrose | 4 mg |
| starch | 2 mg |
| talc | 1 mg |
| stearic acid | 0.5 mg |

### Example 6

### Tablet Composition

The sucrose, microcrystalline cellulose and one of the compounds of the invented combination, as shown in Table VI below, are mixed and granulated in the proportions shown with a 10% gelatin solution. The wet granules are screened, dried, mixed with the starch, talc and stearic acid, then screened and compressed into a tablet.

TABLE VI

| INGREDIENTS | AMOUNTS |
|---|---|
| 2,4-difluoro-N-{2-(methyloxy)-5-[4-(4-pyridazinyl)-6- | 10 mg |
| quinolinyl]-3-pyridinyl}benzenesulfonamide (Compound B) | |
| Microcrystalline cellulose | 60 mg |
| sucrose | 5 mg |
| starch | 10 mg |
| talc | 5 mg |
| stearic acid | 2 mg |

While the preferred embodiments of the invention are illustrated by the above, it is to be understood that the invention is not limited to the precise instructions herein disclosed and that the right to all modifications coming within the scope of the following claims is reserved.

We claim:

1. A combination comprising:
(i) a first compound of Structure (I): 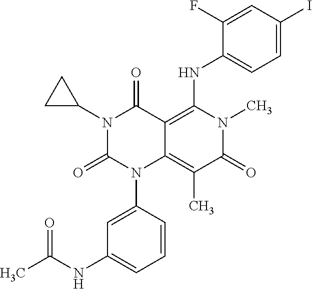
or a pharmaceutically acceptable salt or solvate thereof; and
(ii) a second compound which is represented by Structure (II) 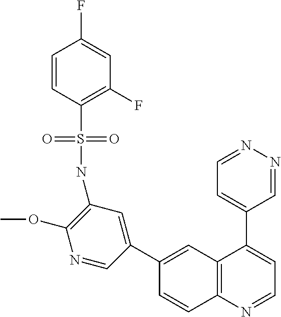
or a pharmaceutically acceptable salt thereof.

2. A combination according to claim 1 wherein the compound of Structure (I) is the hydrate.

3. A combination according to claim 1 wherein the compound of Structure (I) is a solvate selected from the group consisting of: acetic acid, ethanol, nitromethane,
chlorobenzene, 1-pentanol, isopropyl alcohol, ethylene glycol, 3-methyl-2-butanol and dimethyl sulfoxide.

4. A combination according to claim 1 wherein the compound of Structure (I) is the dimethyl sulfoxide solvate.

5. A combination kit comprising a combination according to claim 1 together with a pharmaceutically acceptable carrier or carriers.

6. A combination according to claim 1 where the amount of the compound of Structure (I) or a solvate thereof is an amount selected from 0.125 mg to 10 mg and the amount of the compound of Structure (II) is an amount selected from 0.05 mg to 10 mg.

7. A method of treating cancer in a human in need thereof which comprises administering a therapeutically effective amount of a combination of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof and 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, to a human in need thereof,
wherein the combination is administered within a specified period, and
wherein the combination is administered for a duration of time.

8. A method according to claim 7 wherein the amount of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-[(2H)-yl]phenyl}acetamide, or a pharmaceutically acceptable salt or solvate thereof is selected from about 0.5 mg to about 4 mg and the amount of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, is selected from about 0.5 mg to about 5 mg.

9. A method according to claim 7 wherein the amount of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof is selected from about 0.125 mg to about 3 mg and the amount of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof is selected from about 0.05 mg to about 3 mg.

10. A method according to claim 7 wherein N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and the amount of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, are administered within 12 hours of each other each day for a period of at least 7 consecutive days, optionally followed by one or more cycles of repeat dosing.

11. A method according to claim 7 wherein N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, and the amount of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, are administered within 24 hours of each other each day for a period of at least 7 consecutive days, optionally followed by one or more cycles of repeat dosing.

12. (canceled)

13. A method of treating cancer in a human in need thereof which comprises administering to the human from about 0.5 to 4 mg of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, once a day from day 1 to day 30, optionally followed by one or more repeating cycles; and periodically administer to the human from about 0.5 mg to 5 mg of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, from day 1 to day 30, optionally followed by one or more repeating cycles.

14. (canceled)

15. A method of treating cancer in a human in need thereof which comprises administering to the human from about 0.5 to 5 mg of 2,4-difluoro-N-{2-(methyloxy)-5-[4-(4-pyridazinyl)-6-quinolinyl]-3-pyridinyl}benzenesulfonamide, or a pharmaceutically acceptable salt or solvate thereof, once or twice a day from day 1 to day 30, optionally followed by one or more repeating cycles; and periodically administer to the human from about 0.5 to 4 mg of N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide, or a pharmaceutically acceptable salt or solvate thereof, from day 1 to day 30, optionally followed by one or more repeating cycles

16. A method of claim 13, wherein 2,4-difluoro-N-{2-(methyloxy)-5-[4-(4-pyridazinyl)-6-quinolinyl]-3-pyridinyl}benzenesulfonamide is administered, once every 2-4 days, optionally followed by one or more repeating cycles.

17. A method of claim 13, wherein 2,4-difluoro-N-{2-(methyloxy)-5-[4-(4-pyridazinyl)-6-quinolinyl]-3-pyridinyl}benzenesulfonamide is administered, once every 5-7 days, optionally followed by one or more repeating cycles.

18. A method of claim 13, wherein 2,4-difluoro-N-{2-(methyloxy)-5-[4-(4-pyridazinyl)-6-quinolinyl]-3-pyridinyl}benzenesulfonamide is administered, once every 8-15 days, optionally followed by one or more repeating cycles.

19. A method of claim 15, wherein N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide dimethyl sulfoxide is administered, once every 2-4 days, optionally followed by one or more repeating cycles.

20. A method of claim 15, wherein N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide dimethyl sulfoxide is administered once every 5-7 days, optionally followed by one or more repeating cycles.

21. A method of claim 15, wherein N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide dimethyl sulfoxide is administered once every 8-15 days, optionally followed by one or more repeating cycles.

22. A method according to claim 13, wherein said cancer is colon, lung, liver, pancreatic or breast cancer.

23.-33. (canceled)

34. A method according to claim 7, wherein N-{3-[3-cyclopropyl-5-[(2-fluoro-4-iodophenyl)amino]-6,8-dimethyl-2,4,7-trioxo-3,4,6,7-tetrahydropyrido[4,3-d]pyrimidin-1(2H)-yl]phenyl}acetamide is administered in the form of dimethyl sulfoxide solvate.
